(19) United States
(12) Patent Application Publication
(10) Pub. No.: US 2003/0135269 A1
(43) Pub. Date: Jul. 17, 2003

(54) Laparoscopic-assisted endovascular/endoluminal graft placement

(76) Inventor: Lee L. Swanstrom, Portland, OR (US)

Correspondence Address: Harris Zimmerman, Esq., Suite 710, 1330 Broadway, Oakland, CA, 94612, US

(21) Appl. No.: 10/053,053

(22) Filed: Jan. 16, 2002

(51) Int. Cl. A61F 2/06

(52) U.S. Cl. 623/1.23; 623/1.11

(57) ABSTRACT

A method and apparatus for repair of AAA uses a graft that is introduced intraluminally and secured through laparoscopic and percutaneous access to the repair site. The arterial graft is a flexible, tubular sleeve that is free of any stent structure. A catheter assembly for delivering the graft includes a removable mechanical expansion assembly that is temporarily expanded to impinge the graft ends against the arterial wall to enable fixation of the graft ends. The fastener assembly for securing the graft includes an inner retention member and a pair of deformable wires extending from the inner retention member. The inner retention member is inserted via needle through the arterial wall and the graft and tension is applied to the wires and the inner retention member pulls the graft end into close impingement with the intimal surface of the vessel.

### BACKGROUND OF THE INVENTION

The present invention relates to an apparatus and method for repairing an anatomic vessel wall or the wall of a hollow organ or duct, such as the esophagus or aorta, particularly in the human body. More specifically, the invention relates to devices and methods for delivering a vessel graft or other graft endovascularly or endoluminally to a placement site, and thereafter securing the graft using laparoscopic or percutaneous techniques.

A notable use for the present invention is with regard to an abdominal aortic aneurysm (hereinafter, “AAA”. AAA is a weakening of the wall of the aorta in the abdominal area. Over 160,000 AAAs are diagnosed annually in the United States; one-quarter of AAAs will eventually rupture and, despite many advances in acute medical care, medical transport, and resuscitation, ruptured AAAs continue to have a 50% mortality rate. Thus AAAs comprise a serious health problem for which, arguably, effective treatment has yet to be developed.

A typical AAA is infrarenal, located below the kidneys and above the bifurcation point where the aorta divides into the iliac arteries. The arterial walls bulge outwardly from their normally generally tubular conformation, the bulging being caused by weakening of the aortic vessel walls. The traditional surgical technique for treating AAA involves excision of the aneurytic tissue and replacement of the tissue with either a synthetic graft or a graft from another portion of the patient's body. This surgical approach involves a large abdominal incision that dissects major abdominal muscle groups and fascia, and total bowel displacement and large disruption of the retroperitoneum, followed by excision of the aneurytic tissue and attachment of the replacement graft to the vessel ends. This involves a traumatic access through a large incision, with attendant blood loss, and recuperation typically involves several days in the hospital's Intensive Care Unit and a week or more in the hospital. The manipulation of the bowel and retroperitoneal dissection may result in prolonged ilius, and other detrimental effects such as hypothermia, coagulation problems, a risk of sexual dysfunction, as well as significant pain and disfigurement from the access incision.

Because of the negative aspects of the otherwise effective open surgical procedure, alternative techniques have been developed in the prior art. An early attempt, transfemoral intraluminal graft implantation for AAA, involved inserting a stent graft through the femoral artery and guiding it to the aneurysm site. Upon proper positioning of the stent graft, the stent was deployed and grafted to the vascular walls of the aorta. The use of stent grafts has decreased patient morbidity and, because of the less invasive nature of the technique used to introduce, deploy, and secure the graft, has significantly reduced the problems involved with the open surgical techniques traditionally used for AAA repair. That is, there is less blood loss, less operative pain, a shorter hospital stay, and quicker healing of the smaller incisions.

An alternative to open AAA resection is the use of laparoscopic techniques to accomplish the same goal of excising the aneurysm but avoiding the large abdominal incision. Laparoscopic AAA repair has been described in the surgical literature for several years but has failed to gain widespread acceptance due to its extreme technical difficulty and the low safety margin placement.

It has been proposed to combine the best aspects of the two approaches. Laparoscopic assisted stent-graft placement has been advocated to resolve the problems inherent in both stent-graft placement and fixation.

Although the use of minimally invasive surgical techniques for fixation of the graft have greatly improved AAA repair procedures, this combination is not free of problems. The fixation of the stent graft within the AAA has engendered complications. One technique for securing a stent graft employs hook-shaped projections extending from the stent at proximal and distal ends and disposed to mechanically grip the interior surface of the vascular wall. These hooks may fail to engage properly, or loosen over time, resulting in migration of the stent graft and failure of the AAA repair. Another approach is to employ hook-shaped retaining elements inserted through a band or bracket at the external surface of the aorta to engage the stent body. Moreover, stent grafts themselves have been shown to have their own drawbacks. The infrarenal aorta is subject to rotation and torsional forces as the upper body rotates with respect to the pelvic girdle, but a stent graft by its very rigidity and stiffness is not capable of accommodating rotational motion. Thus a stent graft secured in a AAA repair is subject to rotational movement, and there is ample opportunity for the proximal or distal graft to loosen in the aorta, resulting in endoleaks that are difficult to access and repair. The presence of the fragile stent structure within the flowing bloodstream also increases the risk of embolization if it should fracture. Likewise, the stent graft may experience kinking, or late migration, or endoleaks, or other failure modes cited by the FDA. Other failure modes listed by the FDA include:

metallic component fracture due to material fatigue;

migration of the endograft due to inadequate proximal fixation;

incidence of type I, II, III, IV endoleaks due to weak radial force and lack of conformability;

endograft wear holes due to graft/suture/metal interaction (metal to fabric wear);

kinking of graft limbs due to migration of the endograft;

loss of complete seal to vessel wall due to the attachment design.

Among other issues in this regard are the high cost of stent grafts. Furthermore, the size of the introducers for current stent grafts are too large for 40% of AAA patient population, so that only 60% of people can benefit from endovascular repair today.

It is apparent that the prior art methodolgy and apparatus for AAA repair are in need of further development and improvement.

### SUMMARY OF THE INVENTION

The present invention generally comprises a method and apparatus for repair of AAA using a non-stented graft that is introduced intraluminally and secured through laparoscopic or percutaneous access to the repair site.

One significant aspect of the invention is the provision of an arterial graft that is formed of a flexible, tubular sleeve that is free of a conventional stent structure within the lumen thereof. The graft may be formed of a biocompatible material that is woven or otherwise formed in a sleeve-like configuration. The proximal and distal ends may be provided with an outwardly turned sidewall portion forming an annular cuff, and the cuff may be reinforced with one or more annular bands. The annular bands may be spring-biased to expand outwardly to aid in impinging the cuff portions on the intimal surfaces of the aorta.

(Note: in the following specification, the term “proximal” is used to refer to a direction closer to the patient's heart, and the term “distal” is used to refer to a direction further from the heart.)

The invention provides a catheter assembly for delivering the graft to the repair site intraluminally. A significant aspect of the catheter assembly is the provision of a mechanical expansion assembly that may be temporarily expanded to impinge the graft ends against the arterial wall to enable fixation of the graft ends. The expansion assembly includes a proximal end cap that is secured to the proximal end of a central flexible strut, and a plurality of peripheral flexible struts arrayed circumferentially (with respect to the axis of the catheter) about the central strut. The proximal ends of the peripheral struts are not secured to the end cap, but are selectively entrained and captured within the end cap. During insertion of the catheter, the peripheral struts extend generally parallel to the central strut in a collapsed (unexpanded) state. At the repair site, the expansion assembly may be selectively dilated within the deployed graft by withdrawing the central strut distally, causing the end cap to impinge on the proximal ends of the peripheral struts and exert compressional forces thereon that urge the peripheral struts to bow radially outwardly.

After fixation of the graft, the central strut may be extended proximally to relieve the compressional forces on the peripheral struts. Indeed, the end cap may be freed of its entrainment of the peripheral struts, and the peripheral struts may be withdrawn distally without the end cap. This latter feature enables the peripheral struts to be withdrawn distally from any incidental entanglement or engagement with the fastener devices that extent through the arterial wall to the graft lumen.

Another important aspect of the invention is the provision of an improved method and apparatus for securing a graft within the lumen of a vessel or hollow organ. The apparatus includes an inner retention member, comprised of a rod-like member having a slight curvature, and at least one, and preferably a pair of deformable wires extending from a medial portion of the inner retention member. The inner retention member is secured within a needle-like delivery device with the wires extending therethrough. The delivery device is adapted to be manipulated and operated by a laparoscopic surgical tool, whereby the needle end may be inserted through the arterial wall and through the cuff of the graft to deliver the inner retention member into the lumen of the graft. Thereafter the needle may be withdrawn, and the inner retention member deployed to impinge on the inner surface of the graft. A laparoscopic tool is then used to twist or wind the wires extending from the inner retention member, whereby tension is applied to the wires and the inner retention member pulls the graft end into close impingement with the intimal surface of the vessel. A plurality of fastener members may be installed to circumscribe the cuff portion of the graft. The inner retention members are oriented generally perpendicular to the axis of the graft, the ends of each inner retention member impinging on the reinforcing bands to distribute the clamping force thereto.

In an alternative embodiment, one or more outer retention members may be employed to distribute the clamping forces on the exterior surface of the vessel. In one embodiment, a curved outer retention member may be assembled to the retention wires, prior to the winding step, so that the curved member disperses the clamping force about the periphery of the vessel. In a further alternative, an outer retention member may comprise an omega-shaped component that substantially, but not totally circumscribes the vessel.

In another aspect, the invention provides a sleeve-like graft that is free of any stent structure within the lumen thereof. The graft is formed of a woven biocompatible material, and is provided with reinforcement that increases the longitudinal stiffness of the graft. The reinforcement may include a plurality of pleats extending longitudinally and formed at the exterior surface of the graft, the pleats being angularly spaced about the circumference of the graft. Alternatively, the reinforcement may comprise one or more struts incorporated in the sidewall of the graft. In another alternative, the graft may be reinforced by the inclusion of wire or reinforcing fibers extending longitudinally in the sidewall of the graft.

It is noted that although the invention is described with reference to repair of AAA, it may be applicable to repair of any body vessel or duct or hollow organ.

### BRIEF DESCRIPTION OF THE DRAWING

FIG. 1A is a schematic view of basic aspects of AAA repair using an intraluminally introduced, laparoscopically affixed stentless graft in accordance with the present invention; FIG. 1B is a schematic cross-section of the human abdomen depicting a possible percutaneous access arrangement for AAA repair.

FIG. 2 is a cross-sectional view of a catheter formed in accordance with the present invention and proximally disposed in the infrarenal aorta or the like vessel.

FIG. 3 is a cross-sectional view as in FIG. 2, showing the end cap extended proximally and the graft partially deployed from the catheter assembly.

FIG. 4 is a cross-sectional view as in FIG. 3, depicting the dilation of the mechanical expansion assembly of the catheter assembly, and a plurality of fastener assemblies securing the proximal end of the graft in annular fashion to the vessel wall.

FIG. 5 is a cross-sectional view as in FIG. 4, showing the end cap released proximally from the peripheral struts of the expansion assembly.

FIG. 6 is a cross-sectional view as in FIG. 5, depicting the proximal ends of the peripheral struts of the expansion assembly withdrawn distally and unfettered by the fastener assemblies extending through the graft.

FIG. 7 is a cross-sectional view following FIG. 6, depicting the catheter of the invention proximally disposed at the distal portion of the infrarenal aorta, with the mechanical expansion assembly dilated to expand the graft distal end, and a plurality of fastener assemblies extending through the graft distal end.

FIG. 8 is a cross-sectional view as in FIG. 7, showing the end cap of the expansion assembly extended proximally to release the proximal ends of the peripheral struts and collapse the expansion assembly.

FIG. 9 is a cross-sectional view as in FIG. 8, showing the catheter assembly withdrawn completely and the distal end of the graft secured annularly to the vessel wall of the distal end of the infrarenal aorta.

FIG. 10 is a side view of a fastener assembly loaded into an endoscopic tool, with the jaw in position to deploy the fastener assembly.

FIG. 11 is a side view as in FIG. 10 in which the fastener assembly is positioned in the endoscopic tool to be driven to pierce the vessel sidewall and graft sidewall.

FIG. 12 is a perspective view of the stentless graft of the present invention.

FIG. 13 is an enlarged cross-sectional view depicting one embodiment of the end arrangement of the graft depicted in FIG. 12.

FIG. 14 is a perspective view of the stentless graft of the present invention.

FIG. 15 is a perspective view of an alternative embodiment of the graft of the invention.

FIG. 16 is a perspective view of an alternative embodiment of the graft, a bifurcated docking graft.

FIGS. 17A and 17B are perspective and end views of a further embodiment of the graft, a longitudinally pleated graft.

FIGS. 18A and 18B are perspective and end views of a further embodiment of the graft, a longitudinally reinforced graft.

FIGS. 19A and 19B are perspective views of one embodiment of the mechanical expansion assembly of the present invention, shown in the collapsed (retracted) disposition and expanded disposition, respectively.

FIGS. 20A and 20B are perspective views of another embodiment of the mechanical expansion assembly, shown in the collapsed (retracted) disposition and dilated (expanded) disposition, respectively.

FIG. 21 is a schematic view showing the placement and fixation of a graft using an external band about the vessel in conjunction with the fastener assemblies.

FIG. 22 is a perspective view of one embodiment of the external band of claim 21.

FIGS. 23A and 23B are partial cross-sectional views of a graft secured within a vessel by a fastener member secured externally, without and with an external band or ring.

FIG. 24 is an enlarged partial cross-sectional view of one embodiment of the graft fastening assembly of the present invention

### DESCRIPTION OF THE PREFERRED EMBODIMENT

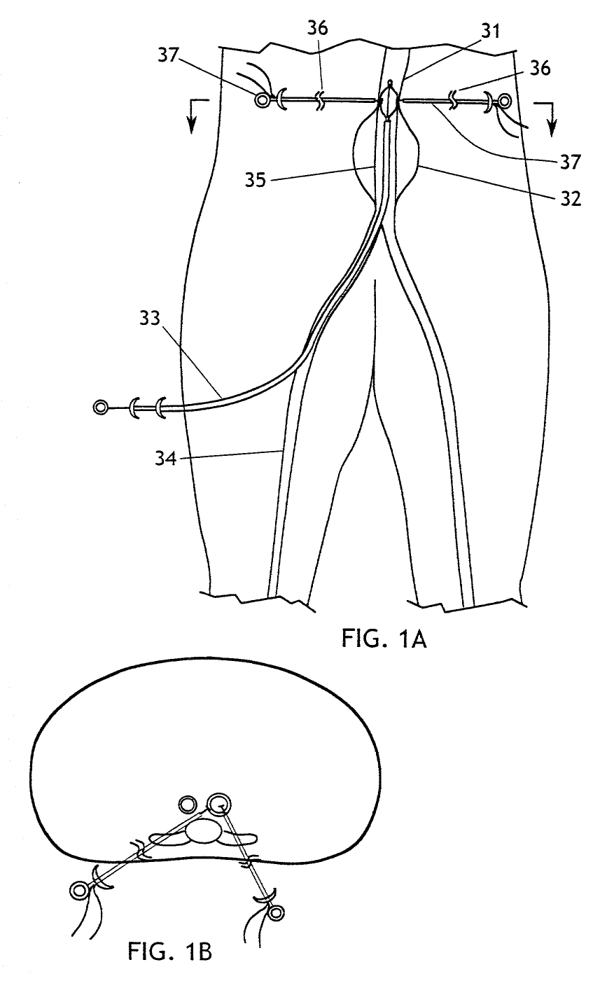

The present invention generally comprises a method and apparatus for delivering a tubular graft assembly to a damaged vessel or hollow body organ, and for expanding and affixing the graft assembly to the wall of the vessel or organ. With regard to FIG. 1A, an anatomic vessel 31, in this case a section of the aorta, presents an aneurysm 32 that is to be repaired. To undertake this repair, a catheter assembly 33 constructed in accordance with the invention is introduced into the femoral artery 34 through a surgical cutdown, and advanced proximally to the aneurysm 32, as is known in the prior art. The catheter assembly transports a graft 35 to the aneurysm site to effect repair thereof. A plurality of access openings 36 are formed in the abdominal wall to provide both visual and mechanical access to the exterior of the vessel 31. A plurality of surgical instruments 37 are inserted through the openings 36 to carry out fixation of the graft to the vessel wall so that the graft acts as an internal shunt to carry blood flow past the aneurysm and prevents the potential hemorrhage thereof.

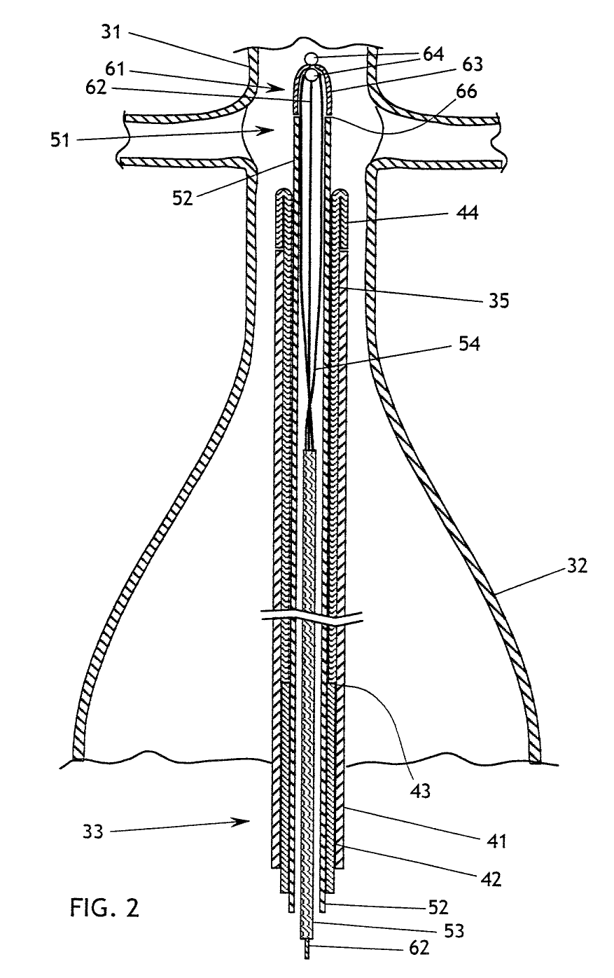

With reference to FIG. 2, the catheter assembly 33 is generally comprised of an outer sheath 41 that is formed of biocompatible material and is flexible yet form-retaining. Disposed concentrically within the sheath 41 is the graft 35, a tubular, sleeve-like component formed of a flexible, expandable, biocompatible material such as woven polymer filament or the like. The graft is positioned at the proximal end of the catheter assembly 33, and a drive tube 42 extends distally from the graft 35 and in end-abutting registration therewith, as shown at reference numeral 43. The graft 35 and tube 42 are slidably disposed within the sheath 41 for selectively independent axial translation therewith. It is noted that the proximal end of the graft 35 includes a cuff portion 44 comprised of the end of the sleeve-like tube of the graft 35 folded retroflexively and distally to impinge on the proximal end of the outer sheath 41. The graft 35 is placed in the sheath 41 in a radially contracted state, so that the catheter is sufficiently small in diameter to pass through the femoral, iliac, and infrarenal aorta arteries without difficulty. The length of the graft 35 is chosen to exceed the length of the aneurysm 32, so that the proximal and distal ends of the graft 35 may be expanded to impinge on healthy vascular wall portions proximally and distally of the aneurysm and be fastened thereto. Further description of the graft construction is given below.

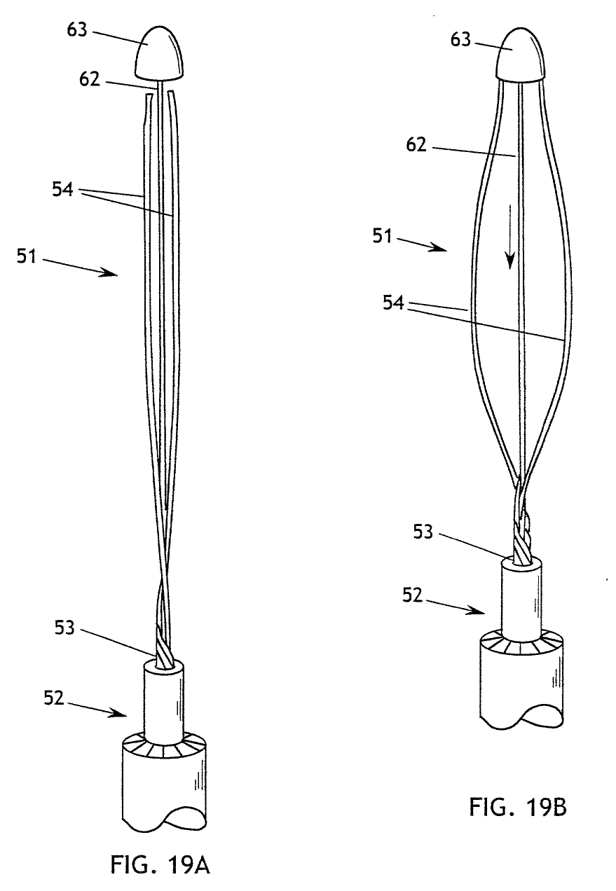

Another significant component of the catheter assembly 33 is a mechanical expansion assembly 51 that is disposed within the lumen of the drive tube 42 and the graft 35. The mechanical expansion assembly 51 is sufficiently flexible to be accommodated within the catheter assembly and to undergo bending together with the outer sheath 41 and drive tube 42, and graft 35. With reference to FIGS. 2 and 19A, the assembly 51 generally includes a flexible confinement tube 52 extending concentrically within the drive tube 42 and dimensioned for selective independent axial translation relative thereto. A flexible strut support 53 extends coaxially through the tube 52, and terminates at its proximal end at a plurality of peripheral struts 54. The struts 54 are flexible and bendable, and may be resiliently biased (sprung outwardly) to expand radially. The proximal ends of the struts 54 are free of attachment, whereas the distal ends are secured to the cable support. The struts 54 are generally arrayed in an angularly spaced apart manner within the confinement tube 52.

The mechanical expansion assembly 51 also includes an end cap assembly 61 extending proximally from the flexible strut support 53. The end cap assembly includes a central strut 62 extending in slidable fashion through the flexible strut support 53, and an end cap 63 is secured to the proximal end of the central strut 62. In this embodiment, the end cap 63 is secured to the strut 62 by a pair of crimps 64 formed on the strut 62 exteriorly and interiorly of the cap to clamp the cap therebetween. The end cap 63 is shown as a bell-shaped structure, but it may have any configuration that exhibits a blunt, convex proximal surface and an annular, concave distal opening that may receive the proximal ends of the peripheral struts, as will be described below.

The end cap 63 and confinement tube 52 are substantially similar in diameter, and are initially disposed in end-abutting relationship, as shown at reference numeral 66. The peripheral struts 54 are retained(confined) in a radially compressed state within the confinement tube 52, and the proximal ends of the peripheral struts may thus be captured within the concave opening of the end cap 63.

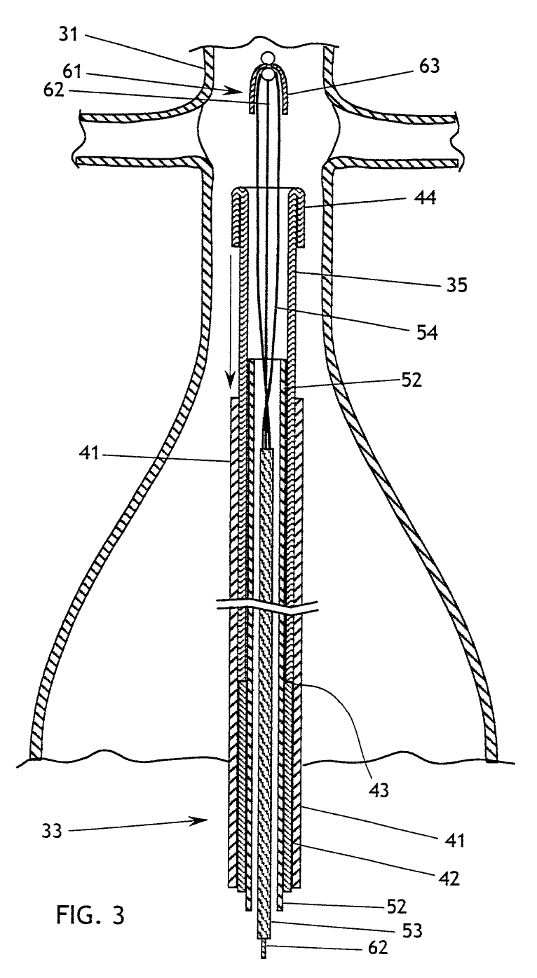

In the initial configuration of the catheter assembly 33 as shown in FIG. 2, the catheter assembly is advanced to the site of the aneurysm 32 to effect repair thereof (This process may involve the use of dilators, a guidewire, an introducer sheath, and other tools and techniques known in the art) The catheter is positioned so that the cuff 44 is positioned in axial alignment with a portion of the vessel proximal to the aneurysm 32. Thereafter, as shown in FIG. 3, the outer sheath 41 is retracted distally to expose a proximal end portion of the graft 35. Note that the position of tube 42 is unchanged, so that the location of the graft 35 remains unchanged as the tube 41 is withdrawn. Likewise, the tube 52 is withdrawn distally to expose the proximal end portions of the peripheral struts 54. Note that the proximal ends of the peripheral struts 54 remain engaged in the end cap 63, the position of which is essentially unchanged.

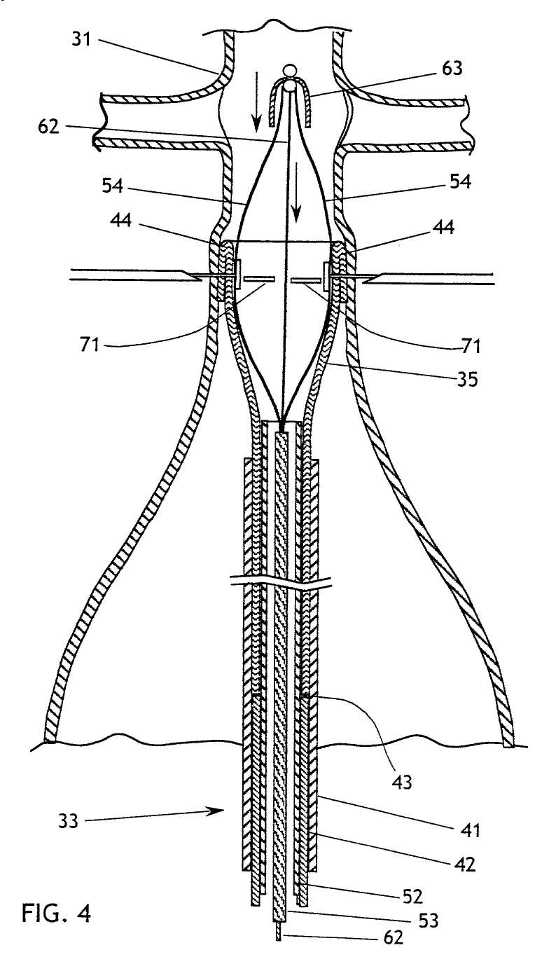

In the next step, shown in FIGS. 4 and 19B, the central strut is withdrawn distally, causing the end cap 63 to axially compress the peripheral struts 54, which bow outwardly in response to the compressive forces applied thereto. The action causes the cuff 44 of the graft 35 to move radially outwardly and impinge forcefully against the intimal surface of the vessel. The cuff 44 is thus expanded and positioned and supported for fastening the cuff to the vessel wall, using fastener assemblies 71 that are described in greater detail in the following specification. The fastener assemblies 71 are introduced through the access ports 36 and installed using laparoscopic or percutaneous surgical tools as described herein. The fastener assemblies are placed annularly about the cuff 44 to form a sealing engagement with the intimal surface of the vessel 31.

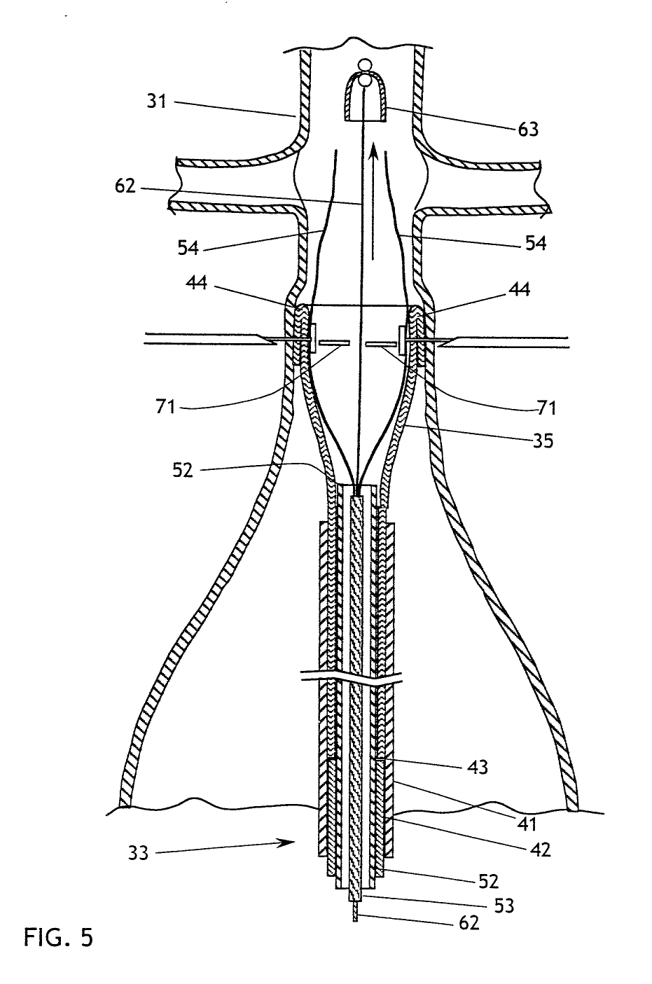

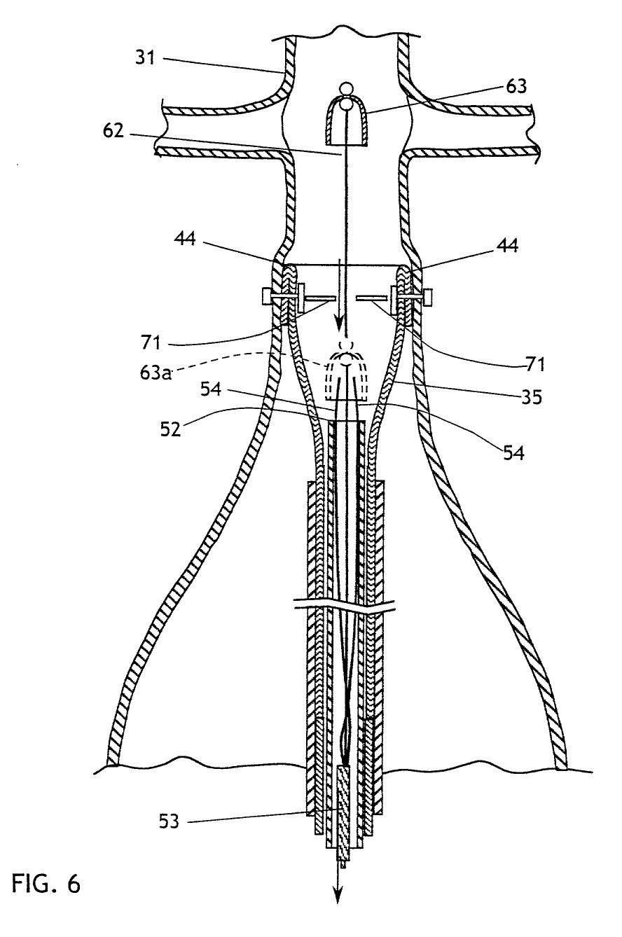

With reference to FIG. 5, the subsequent step involves urging the end cap 63 proximally by pushing the central strut 62 proximally, while at the same time holding the peripheral struts motionless or withdrawing them slightly distally to free the proximal ends of the peripheral struts 54 from the end cap 63. This action releases any compressional force applied from the end cap 63 to the peripheral struts, so that the peripheral struts are not driven to bow radially. In addition, this action enables the peripheral struts 54 to be withdrawn distally, as shown in FIG. 6, by retracting the strut support 53 while the tube 52 remains in place. As a result, the peripheral struts are pulled distally past the fastener assemblies 71 and are freed of any incidental entanglements therewith. In addition, the retraction of the peripheral struts 54 within the tube 52 collapses the peripheral struts 54 radially inwardly to fit the confined diameter of the lumen of tube 52. The consequence of all the steps taken to this point is the fixation of the proximal end of the graft 35 to the interior surface of the vessel 31.

Thereafter, the end cap 63 is retracted distally, as at 63a, so that the concave recess of the end cap is adjacent to the proximal end of the tube 52. The radially confined ends of the peripheral struts 54 are received in the concave recess of the end cap, whereby the end cap 63, struts 54, and tube 52 are returned to approximate the relationship shown in FIG. 2. The entire catheter assembly 33 is then withdrawn distally, with the exception of the drive tube 42, which remains essentially unmoved. The tube 42 holds the graft 35 in its axial position while the remainder of the catheter assembly moves distally, and eliminates tensile forces acting distally on the graft as the catheter withdraws.

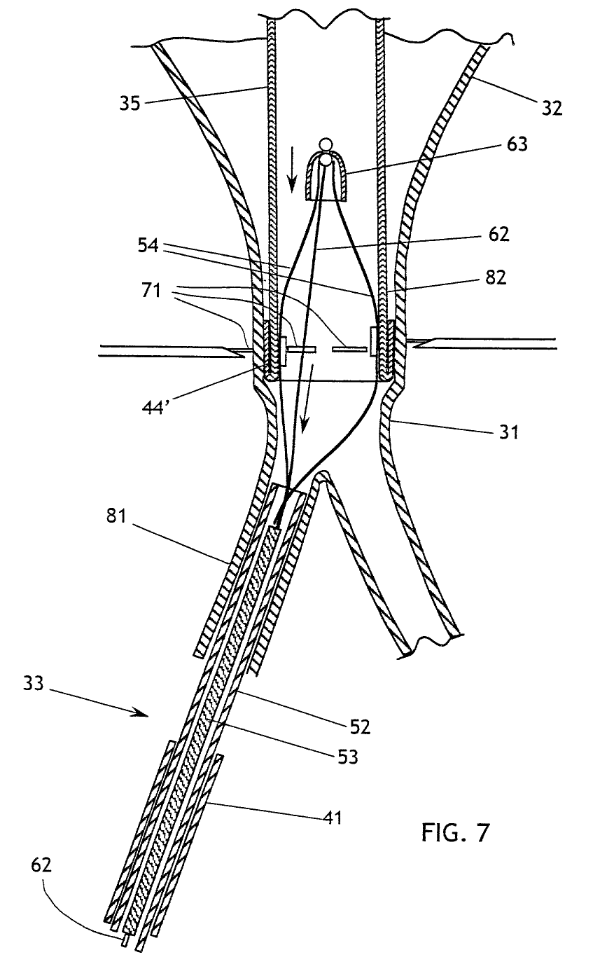

With regard to FIG. 7, the catheter assembly 33 is shown withdrawn distally into a branching vessel 81; e.g., the iliac artery extending from the distal aorta. The distal end 82 of the graft 35 may be provided with a cuff 44′ similar in construction to proximal cuff 44. The mechanical expansion assembly is deployed once again, which involves retracting the tube 52 to expose the struts 54, and then retracting the central strut 62 to cause the end cap 63 to compress the struts 54 axially and expand them radially. The struts 54 thus urge the cuff portion 44′ of the graft 35 against the intimal surface of the vessel 31, and remain in this expanded disposition while a plurality of fastener assemblies 71 are installed through the wall of the vessel 31 and through the cuff 44′. The fastener assemblies 71 are introduced through the access ports 36 and installed using laparoscopic surgical tools and techniques. The fastener assemblies are placed annularly about the cuff 44′ to form a sealing engagement with the intimal surface of the vessel 31.

Thus the graft 35 is completely installed in the vessel 31, forming an internal shunt across the aneurysm 32 that carries blood flow past the diseased portion of the vessel and eliminates the opportunity for hemorrhage.

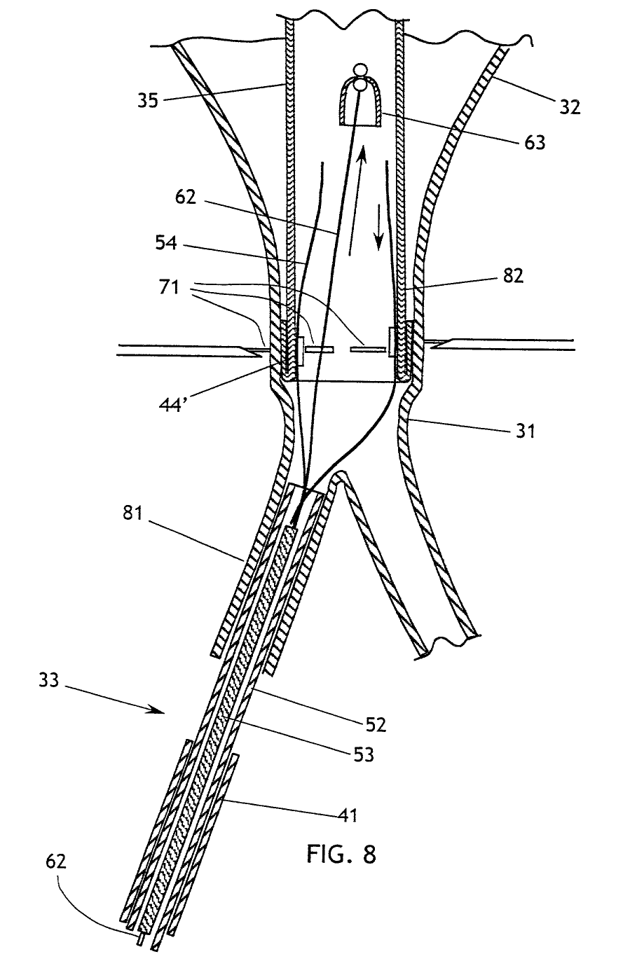

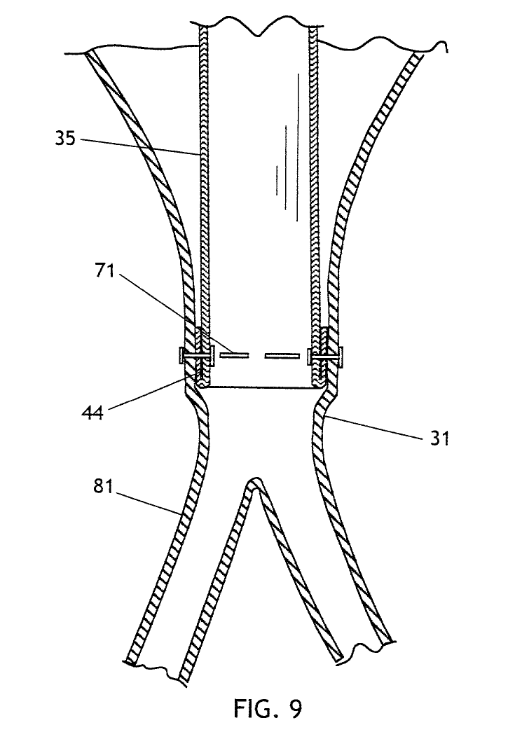

With regard to FIG. 8, the end cap 63 is disengaged from the proximal ends of the peripheral struts 54 by extension of the central strut 62 proximally. The compressional forces acting on the struts 54 are released, and the radial expansion of the struts 54 is significantly diminished. In addition, the proximal ends of the struts 54, by virtue of their lack of attachment to any other component, are free to be withdrawn past the fastener assemblies 71 and freed of any incidental entanglements therewith. This action is carried out by retracting the strut support 53 while the tube 52 remains in place. The struts 54 are thus withdrawn distally into the tube 52, collapsing the struts 54 radially into the lumen of tube 52. Thereafter, the end cap 63 is withdrawn distally by the central strut 62, as depicted previously in FIG. 6, so that the catheter assembly 33 is in condition to be withdrawn completely from the vessels 31 and 81. The result, as shown in FIG. 9, is a completed aneurysm repair. Note that the graft 35 is free of any internal stent or like mechanical structure or framework, and is comprised of a fabric sleeve that is sufficiently flexible to be capable of torsional motion and bending, yet which is sufficiently stiff to resist kinking or collapsing during such flexure.)

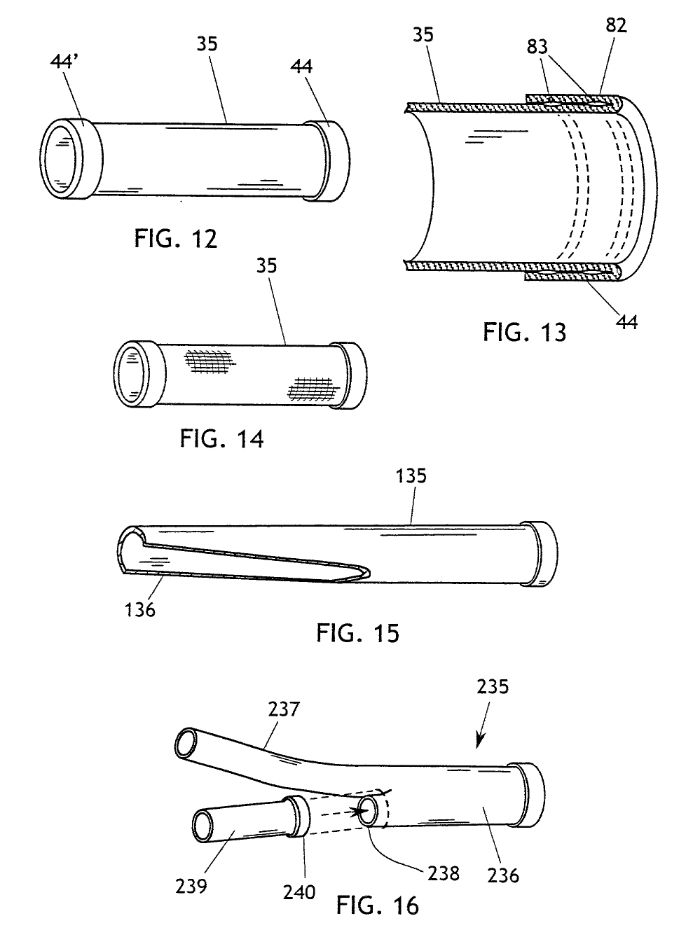

Although the graft of the invention is depicted as comprising a tubular sleeve with cuffs 44 and 44′ at opposed ends, the cuffs should be considered additional improvements to the essential tubular sleeve graft. As shown in FIGS. 12 and 13, each cuff 44 and 44′ includes an end portion 82 folded retroflexively, and at least one, and preferably a pair, of annular bands 83 are secured between the graft body and the folded end portion 82. The bands provide reinforcement to the cuff structure, and also serve to distribute the compressive forces applied to the graft by the fastener assemblies 71. It is preferable to install the fastener assemblies between the axial span of the two bands 83.

Furthermore, the annular bands 83 may be formed of a structure that retains radial elastic compression, whereby the bands 83 tend to expand radially when the cuff 44 is released from the outer sheath 41, as shown for example in FIG. 4. One example of this structure is an annular wire spring or the like, or shape memory alloy components formed in accordance with known techniques to promote radial expansion. As suggested in FIG. 14, the graft 35 is preferably formed of a fabric woven in a tubular configuration and designed to undergo sufficient radial expansion to enable the graft to be transported through a catheter in a collapsed state and expanded, as described above, to engage the sidewall of the vessel.

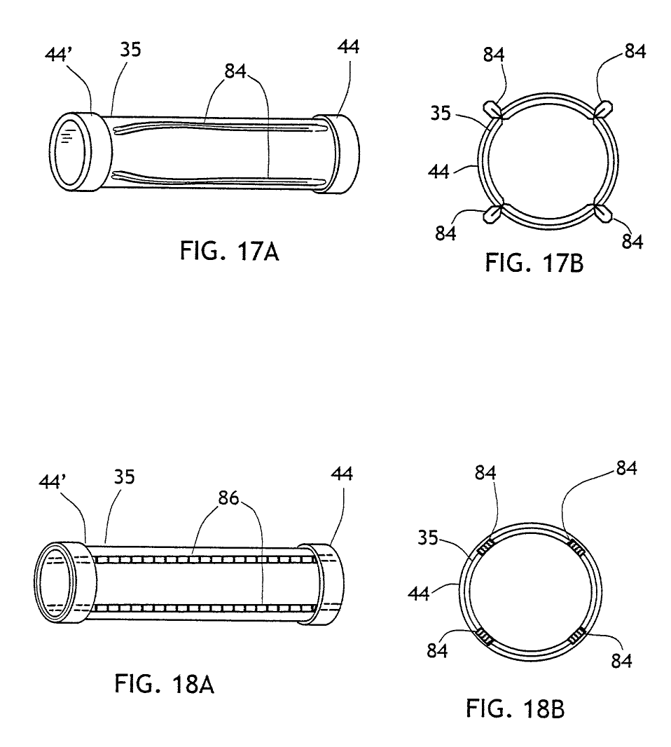

With regard to FIGS. 17A and 17B, the graft 35 may be provided with a plurality of pleats 84 formed in the sidewall of the graft and extending longitudinally therealong. The pleats are disposed at essentially equal angles about the periphery of the graft body, and may be secured by sutures extending longitudinally through the gathered sidewall portions, or by thermal or ultrasonic welding of the sidewall material at the gathered portions, or the like. The pleats are provided to enhance the longitudinal stiffness of the graft body. This increased stiffness aids in resisting the outward pressure of the blood flow through the graft, and resists kinking of the graft under torsion or bending forces. It also assists in the process of deploying the graft to its full length within the vessel or hollow organ.

As shown in FIGS. 18A and 18B, the graft 35 may be augmented with a plurality of reinforcing struts 86 joined to or incorporated within the sidewall of the graft 35. The struts 86 may comprise wires or flexible rods interwoven in the fabric of the graft body or integrally molded into the graft sidewall. Like the pleats described previously, the struts 86 provide increased longitudinal stiffness to the graft body, and the attendant benefits described above.

The graft component of the invention may be provided in many different configurations to suit the range of structural formations in which a graft may be installed. For example, as shown in FIG. 15, the graft 135 may comprise a tubular flexible component having a distal, tapered cutout 136. The graft 135 may be reinforced, if required, by preferably providing a plurality of pleats, as shown in FIG. 17. With regard to FIG. 16, the invention provides a bifurcated graft 235 that is comprised of a flexible tubular body 236 terminating in a split distal end: one elongated tubular leg 237 and one short connector leg 238. This configuration is shaped to extend through the infrarenal aorta to the iliac arteries, the leg 237 extending into the iliac artery through which the catheter 33 introduces and deploys the graft 235. Thereafter, another similar catheter is used to introduce and deploy graft extension 239 through the other iliac artery, the end 240 of the extension 239 being shaped to circumscribe and retain the connector leg 238. This arrangement is designed for situations in which the infrarenal artery does not have sufficient healthy vessel wall to secure any of the grafts described previously.

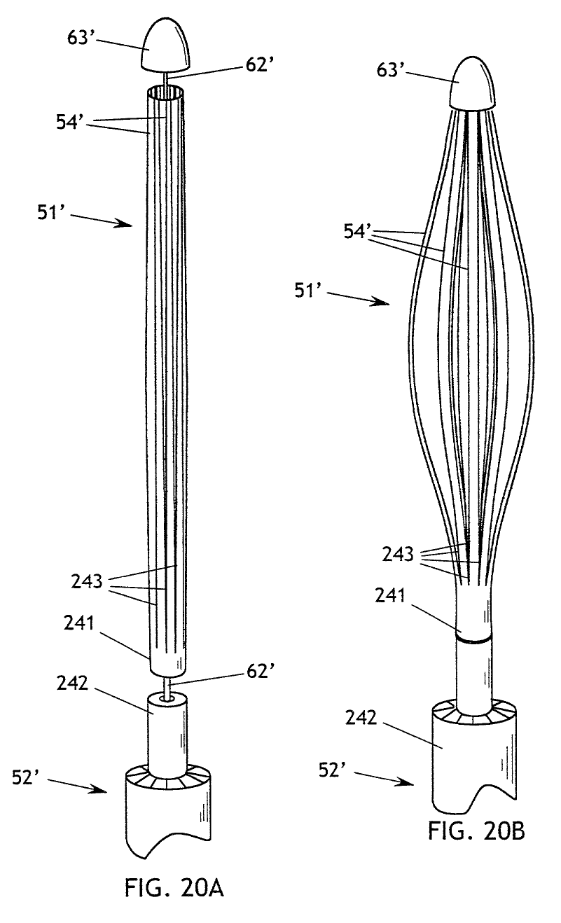

With regard to FIG. 20A, there is shown in isolated view a further embodiment of the mechanical expansion assembly 51′ of the invention that differs in structure, but not function, from the general description of the assembly 51 given previously and shown in FIGS. 19A and 19B. The end cap 63′ is secured to a central strut 62′ by welding or other techniques, and crimp structures are absent. A tube 241 is received about the central strut 62′, and is provided with a plurality of slits 243 extending from the proximal end of the tube 241 to a point adjacent to the distal end thereof. The slits 243 are spaced angularly and disposed to define a plurality of peripheral struts 54′. Each strut 54′ thus comprises a longitudinally extending strip portion of the sidewall of the tube 241, the struts 54′ being arrayed in the circumference of the tube 241. The strut 62′ extends coaxially through a thrust tube 242 in slidable fashion, and the tube 242 is itself slidably disposed within a concentric outer tube 52′.

As shown in FIG. 20B, the assembly 51′ is expanded by retracting the central strut 62′ while also advancing the tube 242 to abut the distal end of tube 241, whereby the struts 54′ are placed in compression between the end cap 63′ and the thrust tube 242. The struts 54′ are thus driven to bow radially outwardly, defining a dilated outer diameter that is significantly greater than the collapsed diameter shown in FIG. 20A. This expansion effect is exploited to support the graft end 44 or 44′ as described above. Note that the tubes 241 and 242 may be withdrawn distally within the tube 52′ to retract the assembly 51′ when it is not in use. The tube 241 (and struts 54′) may be fabricated from a shape memory alloy (SMA) or stress-induced martensitic (SIM) material, as described for example in U.S. Pat. No. 5,067,957, to enhance the expansion capacity of the struts 54′.

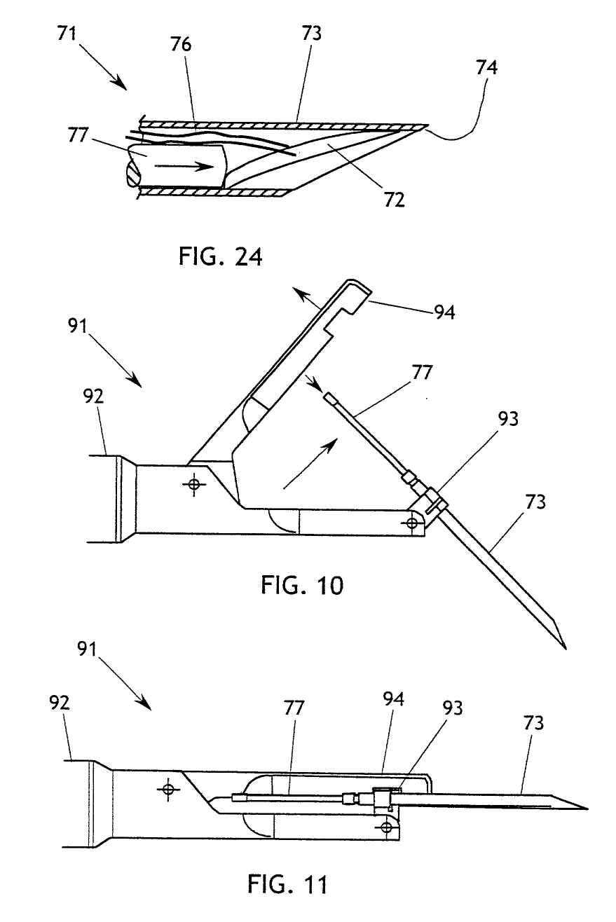

With reference to FIGS. 10, 11, and 24, the fastener assemblies 71 described previously may be comprised of an internal fastener member 72, which is a thin, rod-like component formed of a biocompatible material. The member 72 may be provided with a slight longitudinal curvature, or may be resiliently biased to assume a longitudinally curved configuration in a relaxed state. The member 72 is received within the lumen of a needle 73 having a sharp, piercing end 74. At least one, and preferably a pair of flexible tie connectors such as wires 76 are secured to a medial portion of member 72, the wires extending distally through the lumen of the needle. A push rod 77 is also disposed within the lumen of the needle 73 with sufficient clearance to be slidably disposed with respect to the needle and the wires 76.

As shown in FIGS. 10 and 11, an endoscopic surgical tool 91 includes tool body 92 adapted to be extended through a port in the abdominal wall of the patient, as is known in laparoscopic surgery. The tool includes one jaw provided with a pivoting fixture 93 adapted to secure the needle 73 therein, the push rod 77 extending distally from the needle 73. The other, opposed jaw 94 is configured to close over the needle 73 and push rod 77, as shown in FIG. 11, to form a compact assembly that will pass through the surgical port (typically 5 mm or 10 mm diameter) that provides access to the infrarenal aorta or other vessel 31. In the disposition of FIG. 1, the tool 91 may be used to manipulate the needle end 74 to the external surface of the aorta in registration with the cuff 44 or 44′ of the graft of the invention, and may be used to drive the needle end 74 to pierce the vessel wall and graft cuff.

Thereafter, the jaw 94 may be opened, as shown in FIG. 10, and the fixture 93 is rotated to present the distal end of the push rod 77 in approximate opposition to the jaw 94. The jaw 94 may then be operated to drive the pusher rod 77 to discharge the fastener member 72 from the needle 73 into the lumen of the graft, as described previously. The needle 73 is then withdrawn from the graft and vessel, restored to the compact configuration of FIG. 11, and withdrawn from the surgical site. The wires 76 remain, extending outwardly from the puncture in the vessel wall.

As shown in FIG. 23A, the wires 76 may be grasped by another endosurgical tool having pliers-like jaws 75, and the tool may be rotated repeatedly to wrap the wire 76 about the tool. In this manner the wires 76 may be pulled taut, applying significant tensile force to the fastener member 72 and pulling the graft 35 into close abutment with the intimal surface of the vessel 31. The pliers-like tool may then be disengaged, so that the rolled portion of wires 76 remains impinging on the external surface of the vessel 31 to retain the fastener member tightly against the graft 35. The surgeon may employ a simple torque limiting drive mechanism to wind the wires 76, whereby excessive tension on the wires may be prevented. This process is repeated at selected angular locations along an annulus about the vessel periphery, so that the entire circumference is impinged against the internal vessel surface in a sealing engagement.

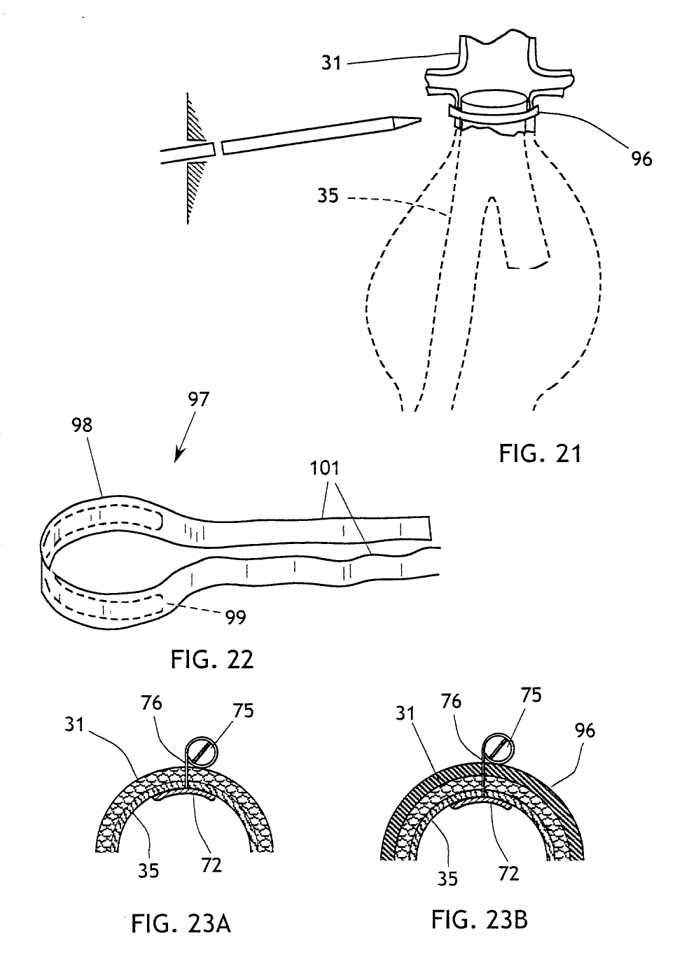

With regard to FIG. 23B, the invention may also provide a curved ring 96 extending about the external surface of the vessel 31. The ring 96, which is curved to conform to the curvature of the vessel wall, is introduced into the abdominal cavity and secured about the vessel 31 prior to installation of the fastener assemblies 71. The needle is driven through the ring 96, vessel 31, and graft 35 to deploy the fastener member 72, as shown in FIG. 21, so that the wires 76 will extend outwardly from the ring 96. Thereafter, the wires 76 are wound or wrapped as described above to place the wires under tension. The tensile force applied by the wires radially inwardly with respect to the fastener member is applied to the ring 96, where it is distributed more uniformly about an annular portion of the vessel wall.

A further embodiment of the ring concept, shown in FIG. 22, provides an omega-shaped member 97 formed of a scrim 98 of flexible material. A reinforcing layer 99 may be applied to the curved portion of the member 97, which is intended to extend entirely about the external surface of the vessel and provide a pressure distribution effect for the wires extending from the fastener members 72. The tails 101 of the member 97 may be trimmed to remove excess amounts after the fastening procedures are completed.

The foregoing description of the preferred embodiment of the invention has been presented for purposes of illustration and description. It is not intended to be exhaustive or to limit the invention to the precise form disclosed, and many modifications and variations are possible in light of the above teaching without deviating from the spirit and the scope of the invention. The embodiment described is selected to best explain the principles of the invention and its practical application to thereby enable others skilled in the art to best utilize the invention in various embodiments and with various modifications as suited to the particular purpose contemplated. It is intended that the scope of the invention be defied by the claims appended hereto.

1. An apparatus for installing an implant in a hollow body organ having a vessel wall, including:
means for transporting said implant into said hollow body organ;
a removable expansion assembly releasably engageable with said implant;
means for dilating said expansion assembly and expanding a portion of said implant against said vessel wall;
means for fastening said portion of said implant to said vessel wall of said organ while said expansion assembly holds said portion against said vessel wall;
means for collapsing said expansion assembly and releasing said portion of said implant.

2. The apparatus of claim 1, wherein said implant comprises a tubular, sleeve-like component.

3. The apparatus of claim 2, wherein said removable expansion assembly is disposed to translate concentrically within said tubular, sleeve-like component.

4. The apparatus of claim 1, wherein said removable expansion assembly includes a plurality of peripheral struts, said struts extending parallel to a longitudinal axis and spaced angularly thereabout.

5. The apparatus of claim 4, wherein said peripheral struts include like proximal ends, said proximal ends being free of mechanical connection.

6. The apparatus of claim 5, wherein said peripheral struts include like distal ends, said distal ends being secured together.

7. The apparatus of claim 5, wherein said removable expansion assembly includes means for compressing said peripheral struts along said longitudinal axis to effect bowing of said peripheral struts radially outwardly from said longitudinal axis.

8. The apparatus of claim 7, wherein said means for compressing includes an end cap, said end cap including means for releasably impinging on said proximal ends of said peripheral struts.

9. The apparatus of claim 8, wherein said removable expansion assembly further includes a central strut extending parallel to said peripheral struts, said central strut being secured to said end cap.

10. The apparatus of claim 9, wherein said means for compressing includes means for translating said central strut distally to urge said end cap to impinge on said proximal ends of said peripheral struts and compress said peripheral struts axially.

11. The apparatus of claim 8, wherein said means for releasably impinging includes a recess formed in a distal surface of said end cap.

12. The apparatus of claim 5, further including means for translating said peripheral struts distally along said longitudinal axis to move said proximal ends of said peripheral struts distally with respect to said means for fastening said portion of said implant to said vessel wall.

13. The apparatus of claim 7, wherein said removable expansion assembly includes a confinement tube, said confinement tube having a lumen dimensioned to receive said peripheral struts in a non-expanded, collapsed state.

14. The apparatus of claim 13, wherein said confinement tube is translatable with respect to said peripheral struts to move said confinement tube selectively into concentric confinement of said peripheral struts.

15. The apparatus of claim 2, wherein said tubular, sleeve-like component includes at least one cuff formed at a proximal end thereof.

16. The apparatus of claim 15, wherein said means for transporting includes a catheter assembly having a first tube.

17. The apparatus of claim 16, wherein said first tube includes a lumen adapted to receive said tubular, sleeve-like component, said first tube having a diameter dimensioned so that the proximal end of said first tube engages said cuff in end-abutting relationship.

18. The apparatus of claim 17, wherein said tubular, sleeve-like component is disposed in said lumen in a radially contracted state.

19. The apparatus of claim 17, wherein said catheter assembly includes a second tube disposed for axial translation concentrically within said first tube, said second tube having a proximal end dimensioned to engage the distal end of said tubular, sleeve-like component in end-abutting relationship.

20. The apparatus of claim 2, wherein said tubular, sleeve-like component includes an axial opening therethrough that is free of any mechanical structure.

21. The apparatus of claim 2, wherein said tubular sleeve-like component includes means for increased longitudinal stiffness.

22. The apparatus of claim 21, wherein said means for increased longitudinal stiffness includes a plurality of pleats extending longitudinally in said tubular, sleeve-like component.

23. The apparatus of claim 21, wherein said means for increased longitudinal stiffness includes a plurality of stiffener struts secured longitudinally in said tubular, sleeve-like component.

24. The apparatus of claim 2, wherein said tubular, sleeve-like component includes at least one cuff formed at one end thereof.

25. The apparatus of claim 24, wherein said at least one cuff includes an end portion of said tubular, sleeve-like component folded retroflexively to impinge on the exterior of said component.

26. The apparatus of claim 25, further including at least one reinforcing band incorporated in said at least one cuff.

27. The apparatus of claim 26, wherein said at least one reinforcing band is resiliently biased to expand radially outwardly.

28. The apparatus of claim 2, wherein said implant has a Y-configuration.

29. The apparatus of claim 28, wherein one branching end of said Y-configuration comprises an elongated tubular leg.

30. The apparatus of claim 28, wherein one branching end of said &-configuration comprises a short connector leg.

31. The apparatus of claim 1, wherein said means for fastening includes a fastener member adapted to be inserted within said implant.

32. The apparatus of claim 31, further including at least one flexible tie connector extending from said fastener member.

33. The apparatus of claim 32, further including needle means for containing said fastener member and flexible tie connector, and means for driving said needle means through the exterior of said vessel wall to pierce said vessel wall and said implant.

34. The apparatus of claim 33, wherein said means for driving includes an endosurgical tool.

35. The apparatus of claim 33, further including push rod means for discharging said fastener member from said needle mean into the interior of said implant, said at least one flexible tie connector including an external portion extending from said fastener member exteriorly of said vessel wall.

36. The apparatus of claim 35, further including means for applying tensile force to said external portion of said at least one flexible tie connector, whereby said implant and said vessel wall are clamped together between said fastener member and said external portion of said at least one flexible tie connector.

37. The apparatus of claim 36, wherein said means for applying tensile force include means for winding said at least one flexible tie connector about an winding axis.

38. The apparatus of claim 37, wherein said means for winding includes a tool having a torque-limiting mechanism.

39. The apparatus of claim 37, wherein said means for winding includes an endosurgical tool.

40. A removable expansion assembly for dilating a surgical implant within a hollow body organ, including:
a plurality of peripheral struts, said struts extending parallel to a longitudinal axis and spaced angularly thereabout;
said plurality of peripheral struts being removably disposed within said surgical implant;
means for urging said peripheral struts to expand radially outwardly from said longitudinal axis, whereby said surgical implant is dilated.

41. The removable expansion assembly of claim 40, wherein said peripheral struts include like proximal ends, said proximal ends being free of mechanical connection.

42. The removable expansion assembly of claim 41, wherein said peripheral struts include like distal ends, said distal ends being secured together.

43. The removable expansion assembly of claim 41, wherein said means for urging said peripheral struts includes means for compressing said peripheral struts along said longitudinal axis to effect bowing of said peripheral struts radially outwardly from said longitudinal axis.

44. The removable expansion assembly of claim 43, wherein said means for compressing includes an end cap, said end cap including means for releasably impinging on said proximal ends of said peripheral struts.

45. The removable expansion assembly of claim 44, further including a central strut extending parallel to said peripheral struts, said central strut being secured to said end cap.

46. The removable expansion assembly of claim 45, further including means for translating said central strut distally to urge said end cap to impinge on said proximal ends of said peripheral struts and compress said peripheral struts axially.

47. The removable expansion assembly of claim 44, wherein said means for releasably impinging includes a recess formed in a distal surface of said end cap.

48. The removable expansion assembly of claim 40, further including means for translating said peripheral struts distally along said longitudinal axis to move said proximal ends of said peripheral struts distally with respect to said end cap.

49. The removable expansion assembly of claim 40, further including a confinement tube, said confinement tube having a lumen dimensioned to receive said peripheral struts in a non-expanded, radially-collapsed state.

50. The removable expansion assembly of claim 49, wherein said confinement tube is translatable with respect to said peripheral struts to move said confinement tube selectively into concentric confinement of said peripheral struts.

51. A fastening assembly for joining a surgical implant to a hollow body organ having a vessel wall, including:
a fastener member adapted to be inserted within said implant;
at least one flexible tie connector extending from said fastener member;
needle means for containing said fastener member and flexible tie connector, and means for driving said needle means through the exterior of said vessel wall to pierce said vessel wall and said implant;
means for applying tensile force to an external portion of said at least one flexible tie connector, whereby said implant and said vessel wall are clamped together between said fastener member and said external portion of said at least one flexible tie connector.

52. The fastening assembly of claim 51, further including push rod means for discharging said fastener member from said needle mean into the interior of said implant.

53. The fastening assembly of claim 51, wherein said means for driving includes an endosurgical tool.

54. The fastening assembly of claim 51, wherein said means for applying tensile force include means for winding said at least one flexible tie connector about an winding axis.

55. The fastening assembly of claim 54, wherein said means for winding includes a tool having a torque-limiting mechanism.

56. The fastening assembly of claim 54, wherein said means for winding includes an endosurgical tool.

57. The fastening assembly of claim 51, further including pressure distribution means secured to said exterior of said vessel wall, said at least one tie connector extending through said pressure distribution means, said means for applying tensile force being disposed externally of said pressure distribution means.

58. The fastening assembly of claim 57, wherein said pressure distribution means includes a ring having a generally C-shaped configuration.

59. The fastening assembly of claim 57, wherein said pressure distribution means includes a strap having a generally omega-shaped configuration.
